(19) United States
(12) Patent Application Publication
(10) Pub. No.: US 2007/0116694 A1
(43) Pub. Date: May 24, 2007

(54) Inhibitor of interaction of granzyme b with golgin-160

(75) Inventors: Hirofumi Doi, Chiba (JP); Masahiro Ezaki, Chiba (JP); Shoichi Masuda, Kanagawa (JP); Tomoya Miyagawa, Tokyo (JP)

Correspondence Address: GREENBLUM & BERNSTEIN, P.L.C., 1950 ROLAND CLARKE PLACE, RESTON, VA, 20191, US

(73) Assignee: Daiichi Pharmaceutical Co., Ltd., Tokyo (JP)

(21) Appl. No.: 10/544,461

(22) PCT Filed: Jun. 16, 2004
(86) PCT No.: PCT/JP04/08781

§ 371 Date: Jul. 27, 2006

(30) Foreign Application Priority Data
Jun. 18, 2003 (JP) 2003-173238

(51) Int. Cl. A61K 38/54; A61K 38/48

(52) U.S. Cl. 424/94.2; 424/94.63

(57) ABSTRACT

It is an object of the present invention to find a protein interacting with Granzyme B and to provide a means for preventing and/or treating diseases caused by the decomposition of the above protein by Granzyme B. The present invention provides a method of using Golgin-160 as a substrate of Granzyme B; a method for screening an inhibitor of the interaction of Granzyme B with Golgin-160 and/or an inhibitor of the decomposition of Golgin-160 by Granzyme B; various types of agent which comprise an inhibitor of the interaction of Granzyme B with Golgin-160 and/or an inhibitor of the decomposition of Golgin-160 by Granzyme B; and a method for preventing and/or treating various diseases which comprises a step of inhibiting the interaction of Granzyme B with Golgin-160 and/or the decomposition of Golgin-160 by Granzyme B.

### TECHNICAL FIELD

The present invention relates to a method of using Golgin-160 as a substrate of Granzyme B, and also to a method for screening an inhibitor of the interaction of Granzyme B with Golgin 160 and/or an inhibitor of the decomposition of Golgin-160 by Granzyme B. Further, the present invention relates to various types of agents including an inhibitor of the interaction of Granzyme B with Golgin-160 and/or an inhibitor of the decomposition of Golgin-160 by Granzyme B, and also to a method for preventing and/or treating various types of diseases, which comprises a step of inhibiting the interaction of Granzyme B with Golgin-160 and/or the decomposition of Golgin-160 by Granzyme B.

### BACKGROUND ART

It is considered that cytotoxic cells of immune system such as cytotoxic T-lymphocytes (CTL) or natural killer cells (NK) are involved in a developmental cause and/or deterioration of graft rejection, graft versus host disease, various types of autoimmune diseases, various types of allergic diseases, and other diseases (Michele Barry et al., “Cytotoxic T lymphocytes: All roads lead to death” in Nature Reviews/Immunology, 2: 401-409 (2002); Pere Santamaria, “Effector lymphocytes in autoimmunity” in Current Opinion in Immunology, 13: 663-669 (2001)).

The cytotoxic mechanism of these cells has broadly been divided into two types. One of the two types is granzyme family which is a system that depends on a plurality of serine proteinases and performs. It has been known that four types of universal granzymes exist in cytoplasmic granules of human cytotoxic cells. Among them, it has been reported that Granzyme B exists at a high abundance and efficiently induces apoptosis into target cells when it acts with the combination of perforin (Michele Barry et al., “Cytotoxic T lymphocytes: All roads lead to death” in Nature Reviews/Immunology, 2: 401-409 (2002)). It has also been reported that when pathologic conditions of graft rejection, graft versus host disease, or autoimmune disease are formed, Granzyme B or perforin is increasingly generated in cytotoxic cells of immune system that infiltrate in target tissues (Jurgen Strehlau et al., “Quantitative etection of immune activation transcripts as a diagnostic tool in kidney transplantation” in Proc. Natl. Acad. Sci. USA, 94:695-700 (1997)); and many other publications).

It has been reported that Granzyme B entering in target cells cleaves procaspase-3 (and several other procaspases), Bid, ICAD (an inhibitor of caspase-activated DNase), or the like, and that it thereby causes activation of a caspase pathway, release of cytochrome C from mitochondria, amplification of caspase activation based on such release, cleavage of DNA due to activation of CAD (caspase-activated DNase), etc., so that it induces apoptosis (Michele Barry et al., “Cytotoxic T lymphocytes: All roads lead to death” in Nature Reviews/Immunology, 2: 401-409 (2002); and Pere Santamaria, “Effector lymphocytes in autoimmunity” in Current Opinion in Immunology, 13: 663-669 (2001)). It has been found that PARP (poly ADP-ribose polymerase), DNA-PKcs (a catalytic subunit of DNA-dependent protein kinase), NuMA (a nuclear mitotic apparatus protein), filamin, proteoglycan, nuclear lamins, or the like can also be a substrate of Granzyme B. However, the biological significance of cleavage or decomposition of these components has not yet been clarified (Michele Barry et al., “Cytotoxic T lymphocytes: All roads lead to death” in Nature Reviews/Immunology, 2: 401-409 (2002)).

On the other hand, it has been reported that Golgin-160 is a protein that is localized in the membrane of the Golgi apparatus, and that the cleavage and release of several tens of amino acids at the N-terminal side thereof promotes the decomposition of the Golgi apparatus during apoptosis, although the role of the protein has been still unknown (Marie Mancini et al., “Caspase-2 is localized at the Golgi complex and cleaves golgin-160 during apoptosis” Int J Cell biol. 149: 603-612 (2000)).

### DISCLOSURE OF THE INVENTION

It is an object to be solved by the present invention to find a protein interacting with Granzyme B and to provide a means for preventing and/or treating diseases caused by the decomposition of the above protein by Granzyme B.

The present inventors have conducted intensive studies directed towards achieving the aforementioned object. The present inventors have first predicted by in silico analysis that Golgin-160 would be a candidate protein interacting with Granzyme B. Subsequently, they have conducted an in vitro experiment, so as to confirm the interaction of Granzyme B with Golgin-160. That is, they have demonstrated that Golgin-160 is decomposed by Granzyme B as a result of such interaction. The present invention has been completed based on these findings.

Thus, the present invention provides a method of using Golgin-160 as a substrate of Granzyme B.

Further, the present invention provides a method for decomposing Golgin-160, which comprises a step of allowing Granzyme B to come into contact with Golgin-160.

Moreover, the present invention provides a method for screening an inhibitor of the interaction of Granzyme B with Golgin-160 and/or an inhibitor of the decomposition of Golgin-160 by Granzyme B, which comprises a step of allowing Granzyme B to come into contact with Golgin-160 in the presence of a test substance.

Further, the present invention provides a reagent kit, which comprises Granzyme B and/or a gene encoding the same, and Golgin-160 and/or a gene encoding the same.

Still further, the present invention provides an inhibitor of the interaction of Granzyme B with Golgin-160 and/or an inhibitor of the decomposition of Golgin-160 by Granzyme B, which are obtained by the above-described screening method of the present invention.

Still further, the present invention provides an apoptosis inhibitor, which comprises an inhibitor of the interaction of Granzyme B with Golgin-160 and/or an inhibitor of the decomposition of Golgin-160 by Granzyme B.

Still further, the present invention provides a graft rejection inhibitor, which comprises an inhibitor of the interaction of Granzyme B with Golgin-160 and/or an inhibitor of the decomposition of Golgin-160 by Granzyme B.

Still further, the present invention provides a medicament for preventing and/or treating diseases caused by the decomposition of Golgin-160, which comprises an inhibitor of the interaction of Granzyme B with Golgin-160 and/or an inhibitor of the decomposition of Golgin-160 by Granzyme B.

With regard to the above-described medicament, diseases caused by the decomposition of Golgin-160 are preferably graft versus host disease, autoimmune disease, or allergic disease.

In addition, the present invention provides a method for inhibiting apoptosis, which comprises a step of inhibiting the interaction of Granzyme B with Golgin-160 and/or the decomposition of Golgin-160 by Granzyme B.

Moreover, the present invention provides a method for inhibiting graft rejection, which comprises a step of inhibiting the interaction of Granzyme B with Golgin-160 and/or the decomposition of Golgin-160 by Granzyme B.

Furthermore, the present invention provides a method for preventing and/or treating diseases caused by the decomposition of Golgin-160, which comprises a step of inhibiting the interaction of Granzyme B with Golgin-160 and/or the decomposition of Golgin-160 by Granzyme B.

With regard to the above-described method, diseases caused by the decomposition of Golgin-160 are preferably graft versus host disease, autoimmune disease, or allergic disease.

### BRIEF DESCRIPTION OF THE DRAWINGS

FIG. 1 shows the results of a local alignment between Granzyme B (referred to as GZMB in FIG. 1) and Golgin-160 (referred to as GOLGA3 in FIG. 1). The amino acid sequences described in FIG. 1 are shown in SEQ ID NOS: 2 to 7 in the sequence listing.

FIG. 2 shows the results of an in vitro protease assay.

A represents TRX-golin-160, B represents procaspase-3, and C represents TRX-LAG 3.

Lane 1 shows the experimental results obtained in the absence of Granzyme B, and lane 2 shows those obtained in the presence of Granzyme B. The arrow indicates a full-length protein.

FIG. 3 shows the decomposition of Golgin-160 by Granzyme B. TRX-Golgin-160-FLAG was incubated at 37° C. for 2 hours in the absence of Granzyme B (lane 1) and in the presence of Granzyme B (lane 2). Thereafter, 2×SDS sample buffer was added at an equivalent amount to the reaction solution, and the mixture was then heated for 5 minutes. Thereafter, the resultant product was separated by SDS-PAGE, and Western blotting was then carried out using an anti-FLAG M2 antibody (Sigma-Aldrich). A band corresponding to the arrow was cut out, and was then subjected to N-terminal amino acid sequence analysis.

### BEST MODE FOR CARRYING OUT THE INVENTION

1. Golgin-160 as a Substrate of Granzyme B

In the present invention, the interaction of Granzyme B with Golgin-160 was predicted by in silico analysis. Specifically, the amino acid sequence of Granzyme B was divided into oligopeptides each having a certain length. Then, a protein having the amino acid sequence of each oligopeptide or an amino acid sequence that is homologous with the above amino acid sequence was searched in the database. Thereafter, a local alignment was carried out between the obtained proteins and Granzyme B. It was predicted that proteins showing a high score in the local alignment would interact with Granzyme B. As a result of such prediction, it was found that oligopeptides IQEAK, VAQVR and ALQSLRL, which are homologous with oligopeptides LQEVK, KAQVK and AVQPLRL consisting of amino acid residues derived from Granzyme B, are present in the amino acid sequence of Golgin-160 that is a protein existing on the membrane of the Golgi apparatus that is an organella involved in the progression of apoptosis. From these results, it was predicted that Golgin-160 interacts with Granzyme B. In the present specification, the term “interaction” is used to mean that two components act on each other or affect each other, such that they bind to each other or that they have a relationship of a substrate and an enzyme.

In Example 1 of the present specification described later, Golgin-160 was identified as a candidate substrate of Granzyme B by the above-described in silico analysis. However, other candidates that are predicted as substrates of Granzyme B by the above-described in silico analysis may also be used in the present invention.

Subsequently, it was confirmed by an in vitro experiment that Golgin-160 is decomposed by Granzyme B as a result of the interaction of Granzyme B with Golgin-160. Such an in vitro confirmation experiment can appropriately be carried out by persons skilled in the art according to the method described in Example 2 of the present specification or methods equivalent thereto.

Accordingly, the present invention provides a method of using Golgin-160 as a substrate of Granzyme B. As an example of this method, Granzyme B is allowed to interact with Golgin-160, so as to decompose Golgin-160. According to the present invention, Golgin-160 has been identified as a novel substrate of Granzyme B for the first time. According to the present invention, it has been found that Golgin-160 can be a substrate of Granzyme B. Thus, it becomes possible to provide a means for preventing and/or treating diseases caused by the decomposition of Golgin-160 by Granzyme B.

2. Screening Method and Reagent Kit

It was found that Golgin-160 is a substrate of Granzyme B and is decomposed by the action of Granzyme B. As a result, it became possible to screen an inhibitor of the interaction of Granzyme B with Golgin-160 and/or an inhibitor of the decomposition of Golgin-160 by Granzyme B by allowing Granzyme B to come into contact with Golgin-160 in the presence of a test substance.

In the present invention, the term “method for screening an inhibitor of the interaction of Granzyme B with Golgin-160 and/or an inhibitor of the decomposition of Golgin-160 by Granzyme B” is used to mean a method for identifying an inhibitor of the interaction of Granzyme B with Golgin-160 and/or an inhibitor of the decomposition of Golgin-160 by Granzyme B. Such a method can be carried out, for example, by applying a method of detecting the decomposition of Golgin-160 by Granzyme B that is described in the present specification, using Granzyme B as an enzyme, and using Golgin-160 as a substrate.

The type of a test substance used in the screening method of the present invention is not particularly limited. Any given compound can be used as a test substance. Such a test substance may be a low molecular weight compound, a compound existing in extracts from natural products, a low molecular weight compound library, a phage display library, or a combinatorial library. These components are all included in the scope of a test substance defined in the present specification. Taking into consideration the use as a medicament, a lower molecular weight compound or a compound library of lower molecular weight compounds is preferable as a test substance.

Granzyme B is allowed to come into contact with Golgin-160 in the presence of the aforementioned test substance, so that Granzyme B is allowed to interact with Golgin-160. It is then detected and measured whether or not the presence of the test substance inhibits the interaction of Granzyme B with Golgin-160 and/or the decomposition of Golgin-160 by Granzyme B, so as to screen a substance of interest.

Detection and measurement of the interaction of Granzyme B with Golgin-160 and/or the decomposition of Golgin-160 by Granzyme B can be carried out by the following method, for example. Granzyme B is added to a suitable buffer solution containing Golgin-160 (e.g. 50 mM Hepes-KOH (pH 7.4), 2 mM EDTA, 1% NP-40, 0.1 M NaCl, and 10 mM DTT). Thereafter, the mixture is incubated at 37° C., and the reaction solution is then separated by SDS-PAGE. It is then stained, so that Golgin-160 and a decomposition product of Golgin-160 can be observed. The presence or absence of the generated decomposition product of Golgin-160 and/or the amount thereof are compared between a case where a test substance has been added to the reaction system and a case where no test substances have been added thereto, so as to evaluate the inhibitory activity of the test substance against the decomposition of Golgin-160 by Granzyme B. It is to be noted that detection and measurement of the presence or absence of a decomposition product of Golgin-160 and/or the amount thereof can be carried out by an immunologic method using an antibody specific to the decomposition product, or that such detection and measurement can also be carried out by a physicochemical method such as chromatography.

The present invention also provides a reagent kit. The reagent kit comprises at least Granzyme B and/or a gene encoding the same, and Golgin-160 and/or a gene encoding the same. That is to say, Granzyme B and Golgin-160 may be provided either in the form of proteins (an enzyme and a substrate, respectively), or in the form of genes.

When Granzyme B and Golgin-160 are provided in the form of genes, such a gene is preferably provided in the form of a recombinant expression vector produced by incorporating the gene into an expression vector capable of being expressed in a suitable host. The combination of a host with an expression vector suitable therefor is known to persons skilled in the art. Examples of a host may include bacteria, yeasts, animal cells, and plant cells. Various types of expression vectors that are suitable for these hosts are also known. Thus, persons skilled in the art could select a suitable host and a suitable expression vector, as appropriate.

In order to facilitate detection or purification, or to add other functions, other proteins such as alkaline phosphatase, β-galactosidase, immunoglobulin Fc fragments such as IgG, or glutathione-S-transferase (GST), or peptides such as FLAG-tag or HIS×6-tag, may be added to the N-terminal side or C-terminal side of the genes of Granzyme B and Golgin-160, directly, or indirectly via a linker peptide or the like, according to known genetic engineering methods.

In addition, not only naturally existing wild type proteins or genes, but also mutant proteins, homologous proteins, mutant genes, and homologous genes may also be used as Granzyme B and Golgin-160 in the present invention, as long as an enzyme reaction involving the decomposition of Golgin-160 (substrate) by Granzyme B (enzyme) can be achieved. Such a mutant protein or homologous protein generally has an amino acid sequence comprising a mutation such as a deletion, substitution, addition, and/or insertion of one or several amino acids with respect to the amino acid sequence of a wild type protein, or an amino acid sequence having a certain degree of homology (for example, approximately 70% or more, preferably 80% or more, more preferably 85% or more, further more preferably 90% or more, and particularly preferably 95% or more) with the amino acid sequence of a wild type protein. A method of obtaining a gene encoding the aforementioned mutant protein or homologous protein has been publicly known. Such a gene can be obtained, as appropriate, by the method described in, for example, Molecular Cloning: A Laboratory Manual (Sambrook et al., Cold Spring Harbor Laboratory Press, Cold Spring Harbor, N.Y., 1989), or by methods equivalent thereto.

Using the kit of the present invention, an inhibitor of the interaction of Granzyme B with Golgin-160 and/or an inhibitor of the decomposition of Golgin-160 by Granzyme B can easily be screened.

3. Inhibitor of interaction of Granzyme B with Golgin-160 and/or inhibitor of decomposition of Golgin-160 by Granzyme B, various types of agents comprising the above inhibitor, and method for preventing or treating diseases using the above inhibitor An inhibitor of the interaction of Granzyme B with Golgin-160 and/or an inhibitor of the decomposition of Golgin-160 by Granzyme B, which are obtained by the screening method described in 2 above, are also included in the scope of the present invention. Such an inhibitor is a substance that is selected from the aforementioned test substances as one exhibiting a desired inhibitory activity.

Since the decomposition of Golgin-160 by Granzyme B is involved in the progression of apoptosis, such an inhibitor of the interaction of Granzyme B with Golgin-160 and/or an inhibitor of the decomposition of Golgin-160 by Granzyme B can be used as an apoptosis inhibitor. In addition, since it is likely that the decomposition of Golgin-160 by Granzyme B is involved in the progression of graft rejection, an inhibitor of the interaction of Granzyme B with Golgin-160 and/or an inhibitor of the decomposition of Golgin-160 by Granzyme B can be used as a graft rejection inhibitor. As described above, an inhibitor of the interaction of Granzyme B with Golgin-160 and/or an inhibitor of the decomposition of Golgin-160 by Granzyme B can be used as a medicament for preventing and/or treating diseases caused by the decomposition of Golgin-160. The type of diseases caused by the decomposition of Golgin-160 is not particularly limited. Examples of such a disease may include graft versus host disease, autoimmune disease, and allergic disease. Hereinafter, the aforementioned apoptosis inhibitor, graft rejection inhibitor, and medicament may collectively be called the agent of the present invention at times in the present specification.

The inhibitor of the interaction of Granzyme B with Golgin-160 and/or inhibitor of the decomposition of Golgin-160 by Granzyme B of the present invention is subjected to a test that is generally conducted in the development of medicaments, and then can be provided as a medicament.

The dosage form of the agent of the present invention is not particularly limited, and the agent can orally or parenterally be administered. A compound as an active ingredient may directly be used as the agent of the present invention. However, it is preferably provided in the form of a pharmaceutical composition that contains the compound as an active ingredient and pharmacologically and pharmaceutically acceptable additives for preparation.

Examples of a pharmacologically and pharmaceutically acceptable additive may include an excipient, a disintegrator or disintegration adjuvant, a binder, a lubricant, a coating agent, a pigment, a diluent, a base, a resolvent or solubilizing agent, an isotonizing agent, a pH regulator, a stabilizer, a propellant, and an adhesive. Examples of an agent suitable for oral administration may include a tablet, a capsule, a powder, a parvule, a granule, a solution, and syrup. Examples of an agent suitable for parenteral administration may include an injection, a drop, a suppository, an inhalant, a percutaneous absorbent, eye drops, eardrops, an ointment, a cream pharmaceutical, and a fomentation.

The applied dose of the agent of the present invention is not particularly limited. An appropriate dose can be selected depending on various conditions such as the beneficial effects of an active ingredient, the purpose of treatment or prevention, the age or symptoms of a patient, and the administration route. The applied dose is generally between 0.001 and 1,000 mg per day per human adult.

In addition, inhibition of the interaction of Granzyme B with Golgin-160 and/or inhibition of the decomposition of Golgin-160 by Granzyme B in a living body or in a cell enable inhibition of apoptosis, inhibition of graft rejection, and prevention and/or treatment of diseases caused by the decomposition of Golgin-160. These methods are also included in the scope of the present invention.

Examples of a means for inhibiting the interaction of Granzyme B with Golgin-160 and/or the decomposition of Golgin-160 by Granzyme B may include a method of administering the above-described inhibitor of the interaction of Granzyme B with Golgin-160 and/or inhibitor of the decomposition of Golgin-160 by Granzyme B, and a method of modifying a portion of the amino acid sequence of Granzyme B and administering the obtained mutant (a dominant-negative mutant), which does not have protease activity but has an affinity for Golgin-160 as a substrate that is equivalent to that of the aforementioned Granzyme B.

The present invention will be specifically described in the following examples. However, the scope of the present invention is not limited by the examples.

### EXAMPLES

### Example 1

In Silico Search for Protein Interacting with Granzyme B

A protein interacting with Granzyme B (Granzyme 2, cytotoxic T-lymphocyte-associated serine esterase 1) was predicted according to the prediction method described in International Publication WO01/67299. Namely, the amino acid sequence of Granzyme B was divided into oligopeptides each having a certain length. Thereafter, a protein having the amino acid sequence of each oligopeptide or an amino acid sequence homologous with the above amino acid sequence was searched in the database. A local alignment was carried out between the obtained proteins and Granzyme B. It was predicted that proteins showing a high score in the local alignment would interact with Granzyme B. Herein, a high score in the local alignment was defined as 25.0 or greater, as with the method described in International Publication WO01/67299.

Granzyme B is serine protease that is a cytotoxic granule secreted from NK cells or cytotoxic T lymphocytes. It has been known that Granzyme B catalyzes a reaction in which a molecule involved in apoptosis is used as a substrate, so that it is involved in the progression of apoptosis.

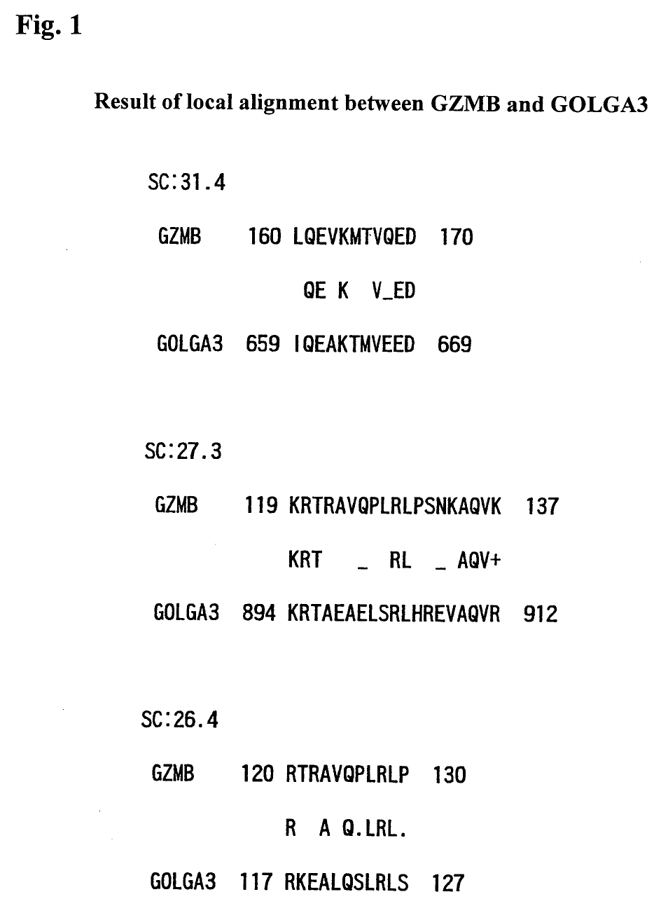

As a result of the prediction, it was found that oligopeptides IQEAK, VAQVR and ALQSLRL, which are homologous with oligopeptides LQEVK, KAQVK and AVQPLRL consisting of amino acid residues derived from Granzyme B, are present in the amino acid sequence of Golgin-160 that is a protein involved in the progression apoptosis and exists on the membrane of the Golgi apparatus which is an organella. FIG. 1 shows the results of the local alignment between Granzyme B (referred to as GZMB in FIG. 1) and Golgin-160 (referred to as GOLGA3 in FIG. 1).

### Example 2

Analysis of Decomposition of Golgin-160 by Granzyme B

An in vitro protease assay was carried out to confirm in an experiment whether or not Golgin-160 is decomposed by Granzyme B.

<Materials>

Construction of Golgin-160 Expression Plasmid

Human Golgin-160 cDNA (the nucleotide sequence is shown in SEQ ID NO: 1 in the sequence listing) was obtained from human lung polyA⁺ RNA by RT-PCR. Substitution and insertion of nucleotides that were probably caused by PCR errors were corrected using Quick Change Multi Site-Directed Mutagenesis Kit (Stratagene). The thus obtained cDNA was then inserted into a pThioHis A vector (Invitrogen) that was an expression vector used for Escherichia coli, to the N-terminus of which ThioRedoxin (TRX)-tag was added, so as to construct a Golgin-160 expression plasmid.

Purification of TRX-Golgin-160

Escherichia coli BL21 Star (DE3) competent cells were transformed with the above-described Golgin-160 expression plasmid. The cells were then cultured at 25° C. overnight in the presence of IPTG (1 mM), so as to allow Golgin-160 to express in the form of an N-terminal TRX fused protein (hereinafter referred to as TRX-Golgin-160). TRX-Golgin-160 was solubilized with a lysis buffer (1% Triton X-100, 1% NP-40, 1% Sarcosyl, 1 mg/ml lysozyme (in PBS)). The obtained solution was dialyzed against 1% Triton X-100 (in PBS), and it was then adsorbed on ProBond Resin (Invitrogen). Subsequently, TRX-Golgin-160 was eluted with imidazole and then dialyzed against PBS. It was then concentrated and used.

Procurement of Granzyme B

Granzyme B, Human, cell culture-derived (Calbiochem, Catalog No. 368042) was purchased, and this product was used as Granzyme B.

Procurement of Procaspase-3

Procaspase-3 was used as a positive control in the in vitro protease assay of Granzyme B. Recombinant Human procaspase-3 (MBL/BioVision, Catalog No. 1083P-5), which had been allowed to express in Escherichia coli, was purchased, and this product was used as such procaspase-3.

Preparation of TRX-LAG 3 (Lymphocyte-activation Protein 3)

TRX-LAG 3 formed by adding TRX-tag to the N-terminus of LAG 3 (lymphocyte-Activation protein 3) was prepared in the same manner as that for TRX-Golgin-160. The prepared TRX-LAG 3 was used as a negative control in the in vitro protease assay of Granzyme B.

<Methods>in Vitro Protease Assay

Granzyme B (0.05 μg) was added to 10 μl of a cleavage buffer containing TRX-Golgin-160, procaspase-3, or TRX-LAG 3 (0.2 μg each) (wherein the buffer consisted of 50 mM Hepes-KOH (pH 7.4), 2 mM EDTA, 1% NP-40, 0.1 M NaCl, and 10 mM DTT) (Kam C. M., Huding D., et al., “Granzymes (lymphocyte serine proteases): characterization with natural and synthetic substrates and inhibitors” in Biochim. Biophys. Acta, 1477:307-323 (2000)). The obtained mixture was incubated at 37° C. for 2 hours. After completion of the incubation, 2×SDS sample buffer (125 mM Tris-HCl (pH 6.8), 4% (w/v) sodium dodecyl sulfate, 20% (v/v) glycerol, 0.01% (w/v) bromophenol blue, and 20% (v/v) 2-mercaptoethanol) was added at an equivalent amount to each reaction solution, and the obtained mixture was heated for 5 minutes. Thereafter, the obtained reaction mixture was separated by SDS-PAGE and then stained with Coomassie Brilliant Blue. Thus, a protein and a decomposition product thereof were observed.

<Results>

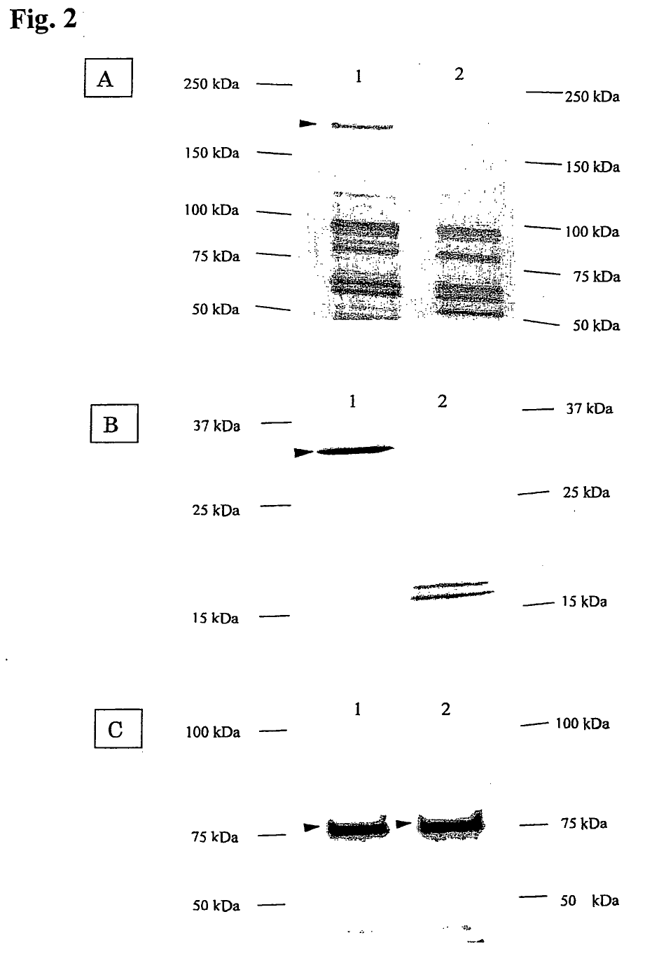

As shown in FIG. 2A, TRX-Golgin-160 was decomposed by Granzyme B. Procaspase-3 as a positive control was decomposed by Granzyme B under the same conditions (FIG. 2B). The N-terminal TRX fused protein NTRX-LAG 3 that had been prepared in the same manner as that for TRX-Golgin-160 was not decomposed (FIG. 2C).

### Example 3

Identification of the Site of the Decomposition of Golgin-160 by Granzyme B

In order to analyze the position of the decomposition of Golgin-160 by Granzyme B, after performing in vitro protease assay, the cleavage fragment was subjected to N-terminal amino acid sequence analysis.

<Materials>

Construction of Golgin-160 Expression Plasmid

The TRX-Golgin-160 expression plasmid prepared in Example 2 was used. A FLAG sequence was inserted into the C-terminus of Golgin-160 cDNA, so as to construct ThioRedoxin-Golgin-160-FLAG (TRX-Golgin-160-FLAG) expression plasmid (pTHIO-HisA/Golgin-160-FLAG).

Purification of TRX-Golgin-160-FLAG

Escherichia coli BL21 competent cells (Novagen) were transformed with the above-described expression plasmid. The cells were then cultured at 37° C. overnight in an LB medium, and they were then cultured at 25° C. for 6 hours in the presence of IPTG (1 mM), so as to allow TRX-Golgin-160-FLAG to express. TRX-Golgin-160-FLAG was solubilized with a lysis buffer (1% Triton X-100, 1% NP-40, 1% Sarcosyl, 1 mg/ml lysozyme in PBS). The obtained solution was dialyzed against 1% Triton X-100 in PBS, and it was then adsorbed on ProBond Resin (Invitrogen). Subsequently, TRX-Golgin-160-FLAG was eluted with imidazole and then dialyzed against PBS. It was then concentrated and used.

Procurement of Granzyme B

A commercially available product was used as Granzyme B as in the case of Example 2.

<Methods>

In Vitro Protease Assay

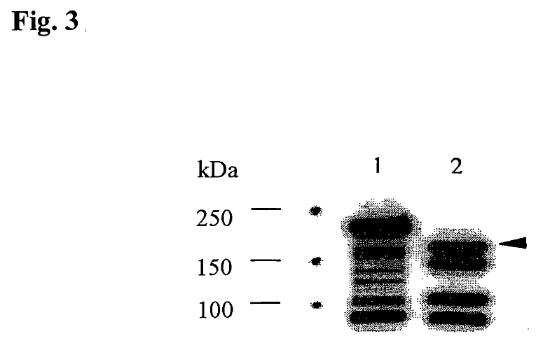

Granzyme B (0.15 μg) was added to 50 μl of a cleavage buffer containing TRX-Golgin-160-FLAG (0.6 μg), (wherein the buffer consisted of 50 mM Hepes-KOH (pH 7.4), 2 mM EDTA, 1% NP-40, 0.1 M NaCl, and 10 mM DTT) (Kam C. M., Hudig D., and Powders J. C., “Granzymes (lymphocyte serine proteases): characterization with natural and synthetic substrates and inhibitors” in Biochim. Biophys. Acta, 1477: 307-323 (2000)). The obtained mixture was incubated at 37° C. for 2 hours. After completion of the incubation, 2×SDS sample buffer was added at an equivalent amount to the reaction solution, and the obtained mixture was heated for 5 minutes. Thereafter, the obtained reaction mixture was separated by SDS-PAGE and then stained with Coomassie Brilliant Blue. As shown in FIG. 3, Western blotting was carried out using an anti-FLAG M2 antibody (Sigma-Aldrich), so as to identify TRX-Golgin-160-FLAG and a decomposition product thereof.

N-terminal Amino Acid Sequence Analysis

Among decomposition products of TRX-Golgin-160-FLAG that had been separated by SDS-PAGE and then had been transcribed on a PVDF membrane, a band having the largest molecular weight was cut out. It was treated with 50% methanol/0.1% TFA and 100% methanol, dried, and then subjected to N-terminal amino acid sequence analysis. Procise cLC 492cLC (Applied Biosystems) was used as a protein sequencer, 140D (Applied Biosystems) was used as a PTH analyzer, and Pulsed-Liquid Prosorb cLC was used as an analysis program.

<Results>

A sequence of 5 amino acids at the N terminus was identified to be Ala-Ser-Pro-Gly-Val (SEQ ID NO: 8 in the sequence listing), which corresponds to positions 93 to 97 of Golgin-160. Accordingly, it was found that Golgin-160 is cleaved by Granzyme B at the position between Asp at position 92 and Ala at position 93.

### INDUSTRIAL APPLICABILITY

In the present invention, it was found for the first time that Granzyme B interacts with Golgin-160 and that Golgin-160 is decomposed by Granzyme B during such interaction. Granzyme B, together with perforin, is secreted from CTL or NK cells, and induces apoptosis in target cells. It is considered that Granzyme B is involved in a developmental cause and/or deterioration of graft rejection, graft versus host disease, various types of autoimmune diseases, various types of allergic diseases, and other diseases. On the other hand, it has been known that Golgin-160 is a protein that is localized in the membrane of the Golgi apparatus, and that the cleavage and release of several tens of amino acids at the N-terminal side thereof promotes the decomposition of the Golgi apparatus during apoptosis. From these facts, the interaction of Granzyme B with Golgin-160 is inhibited, for example, the decomposition of Golgin-160 by Granzyme B is inhibited, so as to prevent and/or treat diseases involving apoptosis promoted by the decomposition of Golgin-160, such as graft rejection, graft versus host disease, various types of autoimmune diseases, or various types of allergic diseases, and other diseases.

1. A method of using Golgin-160 as a substrate of Granzyme B.

2. A method for decomposing Golgin-160, which comprises a step of allowing Granzyme B to come into contact with Golgin-160.

3. A method for screening an inhibitor of the interaction of Granzyme B with Golgin-160 and/or an inhibitor of the decomposition of Golgin-160 by Granzyme B, which comprises a step of allowing Granzyme B to come into contact with Golgin-160 in the presence of a test substance.

4. A reagent kit, which comprises Granzyme B and/or a gene encoding the same, and Golgin-160 and/or a gene encoding the same.

5. An inhibitor of the interaction of Granzyme B with Golgin-160 and/or an inhibitor of the decomposition of Golgin-160 by Granzyme B, which are obtained by the method of claim 3.

6. An apoptosis inhibitor, which comprises an inhibitor of the interaction of Granzyme B with Golgin-160 and/or an inhibitor of the decomposition of Golgin-160 by Granzyme B.

7. A graft rejection inhibitor, which comprises an inhibitor of the interaction of Granzyme B with Golgin-160 and/or an inhibitor of the decomposition of Golgin-160 by Granzyme B.

8. A medicament for preventing and/or treating diseases caused by the decomposition of Golgin-160, which comprises an inhibitor of the interaction of Granzyme B with Golgin-160 and/or an inhibitor of the decomposition of Golgin-160 by Granzyme B.

9. The medicament of claim 8 wherein the diseases caused by the decomposition of Golgin-160 are graft versus host disease, autoimmune disease, or allergic disease.

10. A method for inhibiting apoptosis, which comprises a step of inhibiting the interaction of Granzyme B with Golgin-160 and/or the decomposition of Golgin-160 by Granzyme B.

11. A method for inhibiting graft rejection, which comprises a step of inhibiting the interaction of Granzyme B with Golgin-160 and/or the decomposition of Golgin-160 by Granzyme B.

12. A method for preventing and/or treating diseases caused by the decomposition of Golgin-160, which comprises a step of inhibiting the interaction of Granzyme B with Golgin-160 and/or the decomposition of Golgin-160 by Granzyme B.

13. The method of claim 12 wherein the diseases caused by the decomposition of Golgin-160 are graft versus host disease, autoimmune disease, or allergic disease.


SEQUENCE LISTING (8 sequences)

SEQ ID NO: 1 - DNA, 4593 bp, human
atggacggcgcgtcggccgagcaagatggcctccaggaggacagatcccacagtggcccctcgtctctccccgaggccccactgaagcccccgggcccactggtgccacctgaccagcaggacaaagtccagtgtgccgaggtaaacagagcatccacggaaggggaaagcccggatggacctggccagggaggcctctgtcagaacgggccaacgccacccttcccagaccctccgtcgtctctcgatcccaccacaagcccagtgggccctgatgcctctccaggtgtggctggtttccatgacaacctaaggaagtctcagggaactagtgctgagggcagtgttagaaaagaagctttgcagtctctcagactcagtcttcctatgcaagaaacgcaactgtgctctacagattctcccctgcccctggagaaggaggagcaggtccgacttcaggctcggaagtggctggaagagcagctcaaacagtacagggtgaagcgccagcaggagaggtccagtcaacctgcaaccaaaacgagactttttagcacgcttgatcctgagctcatgttaaacccagaaaacttaccaagggccagtaccctggctatgacaaaagaatattccttcctgcgcaccagtgtccctcgggggcctaaggtgggcagcctggggcttccggcacatcctagggagaaaaaaacttccaaatcaagcaaaatccggtctctggccgattacagaactgaagattcaaatgcggggaattctgggggaaatgtcccggctcccgattctaccaagggttccctgaagcagaacagaagcagtgcggcgtccgttgtgtctgagatcagcctgtcccccgacactgacgaccgtctggagaacacctccctggctggagacagcgtgtctgaggtggatggaaatgacagcgacagctcatcgtacagcagcgcctccacccgagggacctatggcattctgtcgaagacagtgggcacgcaggacaccccctatatggtcaacggccaggagattcctgcggataccctgggccagttcccctccattaaggacgtcctccaggccgcagccgctgagcaccaagaccaggggcaggaggtcaacggggaggtgcggagtcggagagacagcatctgcagcagcgtgtccttggagagctctgcagcagaaacacaggaggagatgctgcaggtgctcaaagagaaaatgcgactcgaaggacagctggaagccttgtcactggaggcgagtcaggcacttaaagagaaggctgagctgcaggcccagctggccgccctcagcacgaagctgcaggcgcaggtggagtgcagccacagcagccagcagcggcaggattcgctgagctcggaggtggacaccctgaagcagtcgtgctgggacctggagcgagccatgactgacctgcagaacatgctggaggcaaaaaatgccagcctggcgtcgtccaacaacgacttgcaggtggccgaggagcagtaccagaggcttatggccaaggtagaggacatgcagaggagcatgctcagcaaggacaacacagtgcacgacctgcgacagcagatgacagccttgcagagccagcttcagcaggtgcagctggagcggacgacgctgaccagcaagctgaaggcgtcgcaggcggagatctcgtccctgcagagtgtccggcagtggtaccagcagcagctcgccctggcacaggaggcccgcgtcaggctgcagggtgagatggcccacatccaggttggacagatgacccaggcaggtctcctggagcacctgaaactcgagaatgtgtccctgtcccagcagctgacggaaactcagcacaggtccatgaaggagaaggggcgcatcgcggcacagctgcagggcattgaggctgacatgttggatcaggaagcagccttcatgcagattcaggaggcaaagacgatggtggaggaggaccttcagaggaggctggaagagtttgaaggtgagagggagcggctgcagaggatggcggactcggcggcatccctggagcagcagctggagcaggtgaagttgactttactccagcgagaccagcagcttgaggctttgcagcaggagcacctggacctgatgaaacagctcaccttgactcaggaggctctgcagagcagggagcagtccctcgatgccctgcagacacactacgatgagctgcaggccaggctgggggagctgcagggcgaggccgcctccagggaggacacgatctgcctcctgcagaacgagaagatcatcttggaggcggctttgcaggcggccaagagtggcaaggaggagcttgacagaggagcaagacgcttggaagaaggtaccgaggaaacgtcggaaactttagagaagttaagagaagaattagctatcaaatccggccaggtggaacacctgcagcaggagactgctgctctgaaaaagcaaatgcaaaaaataaaggaacagtttctccaacaaaaggtgatggtggaggcctaccggcgcgacgccacctccaaagaccagctcatcagtgagctgaaagccaccaggaagaggctggactcggagctgaaggagctgcggcaggagctgatgcaagtgcacggggagaagcggactgccgaggcggagctctcgcgcctgcacagagaggtggcccaggtccgtcagcacatggcggaccttgaagggcatctccagtcggcgcagaaggagcgagacgagatggaaacacacttgcagtcgttgcagttcgataaggagcagatggtcgcggtcacagaggccaatgaggcgctgaagaaacaaatcgaagagttgcagcaagaggcccggaaggccatcacggaacagaagcagaagatgaggcggctgggctcagacttgaccagcgcccagaaggagatgaagaccaaacataaggcctacgagaacgccgtgggcatcctcagccgccgcctgcaggaggccctcgcggccaaggaggctgcggacgcggagctgggccagctccgagcccagggtggcagcagtgacagcagcctggctctacatgaaaggatccaggccctggaggcggagctgcaggctgtcagtcatagcaagacgctgctggaaaaggaactgcaggaggtcatagcgctgaccagccaggagctggaggagtcccgggagaaggtgctggagctggaggacgagcttcaagaatccagaggctttaggaagaagataaaacgccttgaggagtcaaacaagaagttggctcttgaattagagcacgagaaagggaagcttacgggcctcggtcagtccaacgcagctctgcgggaacacaacagcatcctagaaacagctttggccaagagggaggcagacctagtccagttgaaccttcaggtgcaggcagttttgcagcgcaaagaagaggaggatcgccagatgaagcatcttgtccaggccctgcaggcctcactagagaaggagaaggagaaggtgaacagcctcaaggagcaggtggctgctgccaaggtggaagccgggcataaccgccgccacttcaaggcggcctccttggagctgagtgaggtgaagaaggagctgcaggccaaggaacacctggtgcagaagctgcaggccgaggccgacgaccttcagattcgggaggggaaacattcccaggagatagcacagttccaagcagagctggccgaggcccgggcacagctccagctcctgcagaagcagctggacgagcagctcagcaaacagcccgtgggaaaccaagagatggaaaatctcaaatgggaggtggatcagaaagaaagagaaatccagtccttgaagcagcagctggacttgacggagcagcagggcaggaaggaactggaagggctacagcagctgctgcagaacgtcaagtctgagttggagatggcccaggaagacctgtccatgacccagaaggataaatttatgctccaggcaaaagtgtcggagctgaagaacaacatgaagaccctgctccagcagaaccagcagctcaagctggacctacgccgcggccaagacgagaaaggagccgaaagcgcaggccagctcttccaaccctgccacgcccatcaagatcccggactgcccagttcccgcctcgctgctggaggagctgctgagaccaccgcccgccgtgagcaaggagcccctcaagaacctgaacagctgcctccagcagctcaagcaggagatggacagcctgcagcgccagatggaggagcacgccctgacggtgcacgagtctctgtcctcgtggacgccgctggagccagccactgccagccctgtgcccccggggggtcacgccggcccacgcggcgacccacagagacacagtcagagcagggcttccaaagaagggccgggagagtgactgctgtggactcgcctccgtgcgccgctgccccagaaggctcttatcaatgttatttatttgattgtgtcgtcgatgtttttctaagacatgaaatttaa

SEQ ID NO: 2 - protein, 11 aa, human
LQEVKMTVQED

SEQ ID NO: 3 - protein, 11 aa, human
IQEAKTMVEED

SEQ ID NO: 4 - protein, 19 aa, human
KRTRAVQPLRLPSNKAQVK

SEQ ID NO: 5 - protein, 19 aa, human
KRTAEAELSRLHREVAQVR

SEQ ID NO: 6 - protein, 11 aa, human
RTRAVQPLRLP

SEQ ID NO: 7 - protein, 11 aa, human
RKEALQSLRLS

SEQ ID NO: 8 - protein, 5 aa, human
ASPGV